(19) United States
(12) Patent Application Publication
(10) Pub. No.: US 2020/0178900 A1
(43) Pub. Date: Jun. 11, 2020

(54) VESTIBULO-ACOUSTIC SIGNAL PROCESSING

(71) Applicant: NeuralDx Ltd., Toorak, Victoria (AU)

(72) Inventor: Brian John LITHGOW, Toorak, Victoria (AU)

(21) Appl. No.: 16/614,730

(22) PCT Filed: May 18, 2018
(86) PCT No.: PCT/AU2018/050477

§ 371 Date: Nov. 18, 2019

(30) Foreign Application Priority Data
May 18, 2017 (AU) 2017901878

(51) Int. Cl. A61B 5/00; A61B 5/16; A61B 5/04; G16H 40/63; G16H 50/20; G16H 50/70

(52) U.S. Cl. CPC A61B 5/726; A61B 5/16; A61B 5/4839; G16H 50/70; G16H 40/63; G16H 50/20; A61B 5/04

(57) ABSTRACT

A method for processing a vestibulo-acoustic signal, including receiving an vestibulo-acoustic signal obtained from a person; decomposing said signal using wavelets; differentiating said signal and phase data of said wavelets to determine loci of components of a composite field potential waveform produced by the vestibular system of the person.

### FIELD

The present invention relates to vestibulo-acoustic signal processing, and in particular to signal processing methods and systems that are able to isolate components of a vestibulo-acoustic signal obtained from a person to enable diagnosis or to determine drug efficacy in relation to mental disorders.

### BACKGROUND

The diagnosis and treatment of mental disorders can be extremely difficult for clinicians, primarily because it can be difficult to discriminate between conditions that may present with similar symptoms. For example, the American Psychiatric Association produces the Diagnostic and Statistical Manual of Mental Health Disorders (DSM-5) that acts as a manual to define disorders and describe psychopathology. Whilst the manual provides qualitative assessment rating scales that allow qualitative subjective assessments to be made by clinicians (by classifying clusters of symptoms) misdiagnoses can occur due to symptoms varying in their presentation over time, particularly at different times when a patient is assessed.

Accordingly, there is a real need for a reliable system and process that can consistently, sensitively and most of all, objectively measure signals obtained from a person so the person's brain function can be measured in normal and dysfunctional states, and that also allows identification of changes in those states that may be caused by any therapeutic interventions or natural recovery processes. This need has driven the development of the neural event extraction process (“NEEP”) described in International Patent Application No. PCT/AU2005/001330 (“Lithgow 1”), and the subsequent systems and processes described in International Patent Application No. PCT/AU2008/000778 (“Lithgow 2”) and PCT/AU2010/000795 (“Lithgow 3”). This focused on an analysis of signals produced by the vestibular system under various conditions using a technique now referred to as electrovestibulography (EVestG). The electrical vestibulo-acoustic signal obtained from a person is less than a few microvolts and is received with considerable unwanted noise that makes it extremely difficult to extract relevant technical features or components of the signal received. Whilst Lithgow 1, Lithgow 2 and Lithgow 3 describe isolation of relevant components and biomarkers, the systems and processes described have limitations. For example, it can be difficult to isolate those components of a response that derive predominantly from the vestibular system, particularly given the signal to noise ratio of the vestibulo-acoustic response. It can also be difficult to determine precisely the physiologic characteristics of the person that correspond to the field potentials or parts of those potentials that are extracted, including the summing potential (SP). NEEP relies on extracting characteristic peaks of the field potential (FP) waveform that follows a template or model of a characteristic EVestG FP waveform, and the true EVestG FP template or model needs to be better determined and defined.

Also whilst a tilt chair can be used to evoke a vestibulo-acoustic response, it would also be useful to be able to provide equipment that can obtain a useful response in various conditions including whilst a patient is stationary, particularly where it is not possible to move a patient as described in Lithgow 2.

Accordingly, it is desired to address the above or at least provide a useful alternative.

### SUMMARY

An embodiment of the present invention provides a method for processing vestibulo-acoustic signals, including determining major components of a composite vestibulo-acoustic signal produced at least by the vestibular system of a person that respectively relate to a potassium channel for depression, a sodium channel for traumatic brain injury and a third component for discriminating between bipolar disorder and a major depressive disorder. Each major component consists of building blocks, at least one of an excitatory post synaptic potential (EPSP), an internal auditory meatus far field potential (IAMFFP) and an extracellular field potential (EFP). The recorded overall FP from the person includes at least the three major components repeated in a scaled manner before or after a central action potential (AP) region of the response. These correspond to a prior smaller miniature field potentials (FP) (in the 0-7.5 ms range of the response from the person and referred to as pre-components), larger miniature field potentials (in the 1-9 ms range of and referred to as central components), and a post FP smaller miniature field potential (in the 6.5-10 ms range and referred to as post-components). There is also a singular pre-pre component (in the 0-4 ms range). In other words there are composite waveforms (pre-pre, pre, central, post) each incorporating three major component waveforms (efferent, afferent and vestibular nucleus (VN)) and each built up from the three building block waveforms (EPSP, IAMFFP, EFP).

An embodiment of the invention also involves processing the received vestibulo-acoustic signal to obtain interval histogram data, and the intervals between every (particularly the 33^{rd}) detected miniature field potential can be used in discriminating between BD and MDD.

An embodiment of the invention also provides a HMD display presenting images to invoke a response signal from a person, and a double wrapped coaxial cable lead part of recording electrodes attached to the person each with matched impedance. The vestibulo-acoustic response signal may be obtained when a person is in a supine position.

### DRAWINGS

Embodiments of the present invention are described herein, by way of example only, and with reference to the accompanying drawings, wherein:

FIG. 1 is a block diagram of a preferred embodiment of a vestibulo-acoustic processing system;

FIG. 2 is a diagram of the vestibular periphery hair cell to vestibular nucleus to efferent vestibular system (EVS) positive feedback loop of the vestibular system, and a generalised form of the FP waveform produced by the system;

FIG. 3 is a plot of an excitatory post synaptic potential (EPSP) component of the FP waveform;

FIG. 4 is a plot of an extracellular field potential (EFP) component of the FP waveform;

FIG. 5 is a plot of an internal auditory meatus far field potential (IAMFFP) component of the FP waveform;

FIG. 6 is plot of a model of the FP waveform (thickest black line) constructed from major component signals each being composed of one to three building block waveforms, when the components of the model are pre-pre-VN contra, pre-VN1, pre-VN2, pre-VN contra, central-afferent, central VN1, central-EVS, central VN2 components, central-VN contra, post-VN1, post-VN2 and post-afferent;

FIG. 7 shows a plot of development of the model when the only component is the afferent central component;

FIG. 8 shows a plot of development of the model when the components of the model are the pre-VN1, pre-VN2, central-afferent, central VN1 and central VN2 components;

FIG. 9 shows a plot of development of the model when the components of the model are the pre-VN1, pre-VN2, pre-VN contra, central-afferent, central VN1, central-EVS and central VN2 components;

FIG. 10 is a plot of the model of the FP waveform and FP waveform generated by the vestibulo-acoustic processing system;

FIG. 11 is plots of waveforms generated by the vestibulo-acoustic processing system and obtained from normal animals (baseline), deaf animals (dashed line) and animals that are deaf and have been subject to vestibular ablation (dashed and dotted line);

FIG. 12 is a flow diagram of a neural event extraction process executed by a extractor of the vestibulo-acoustic processing system;

FIG. 13 is a plot of average static field potential responses for control, bipolar disorder (BD) and major depressive disorder (MDD) patients produced by the vestibulo-acoustic processing system;

FIG. 14 is an interval histogram (IH) plot for BD and MDD patients produced by the vestibulo-acoustic processing system;

FIG. 15 is interval histograms for patients generated by the vestibulo-acoustic processing system in response to head mounted display images;

FIG. 16 is interval histograms for patients generated by the vestibulo-acoustic processing system in response to head mounted display images and illustrating the effect of different intensities on a 33-Histogram interval for the blue color for the photopic region and mesopic region; and

FIG. 17 is a plot of the model (filtered composite line) incorporating the afferent (EPSP, EFP and IAMFFP) compared with an unfiltered average experimentally recorded composite waveform (unfiltered composite line).

### DESCRIPTION

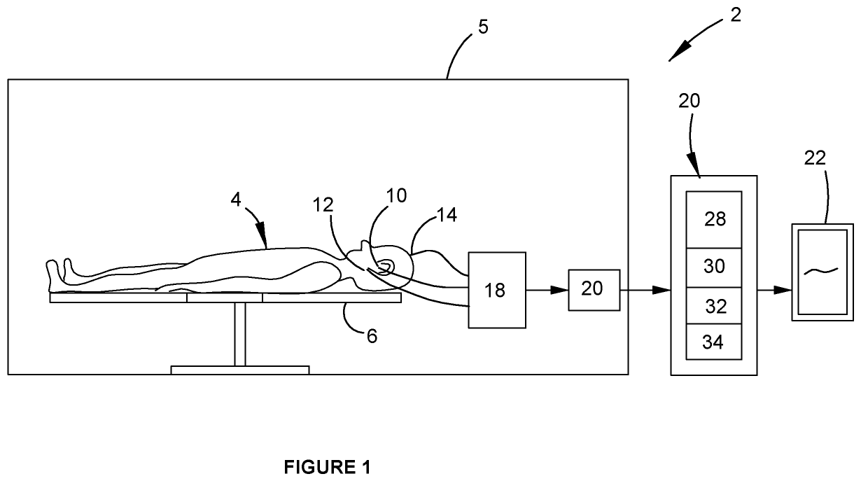

A vestibulo-acoustic signal processing system 2, as shown in FIG. 1, is used to obtain a vestibulo-acoustic signal from a person or patient 4 placed in a sound attenuating booth or testing room 5. The vestibulo-acoustic signal processing system 2 includes a computer system 20 that is normally outside and may be remote from the room 5. The vestibulo-acoustic signal obtained from the patient 4 in the room 5 may be output directly to the computer system 20 for processing or stored for subsequent processing. The computer system 20 includes an analysis module 30 to process the vestibulo-acoustic signals received to produce field potential (FP) data or plots for display on a display screen 22 for a user. Vestibulo-acoustic signal responses can be obtained from the patient 4 using different equipment of the system 2, as described below, and can be obtained spontaneously from the patient 4 or in response to a stimulus.

The vestibulo-acoustic signal is obtained from the person's ear and is done in a manner so it is primarily the product of the vestibular system and hence it can be considered to be an Electrovestibulography (EVestG) signal. To achieve this, a first electrode 10 is placed proximal to the tympanic membrane of an ear of the patient 4 and a second electrode 12 is placed on the patient's ipsilateral earlobe (or outer ear canal), as a reference point. Both electrodes 10 and 12 are the same, and comprise a saline/gel soaked cotton wool electrodes with a double wrapped coaxial cable of matched impedance. The active and reference electrodes 10 and 12 are designed to have matching impedances, and be coaxially electrically shielded. The tips are constructed of cotton wool inpregnated with conductive gel and saline but the tips could be made of other materials, such as Ag-AgCl or graphene coated, substrates of cotton-wool hydrophilic, open pored hydrophilic polyurethane foams, or conductively coated PET flexible film loops or fibers. A third electrode 14 is connected to the forehead of the patient 4. All three electrodes 10, 12 and 14 are connected to an amplifier 18 with the third electrode 14 connected to the common port of the amplifier 18. The impedance is matched between the active or reference and ground electrodes 10, 12, and 14. The amplified vestibulo-acoustic signal obtained from the person 4 is then passed by the amplifier 18 into an analogue digital converter (ADC) 20 so the signal is placed in a digital form for processing by the computer system 20. The electrodes 10, 12 and 14, the amplifier 18 and the ADC 20 may be placed in a set of headphones that the patient 4 is able to wear. A vestibulo-acoustic signal response can be obtained from the patient whilst they are placed in the supine position, as shown in FIG. 1. A signal response can also be obtained in response to a stimulus. The stimulus may be obtained by placing the patient 4 on a chair 6, such as a recliner lounge chair, that allows the patient's head to be tilted involuntarily, as described in Lithgow 1 and Lithgow 2. The stimulus can then be produced using a wide range of different head tilts. Alternatively, the stimulus can be obtained by subjecting the patient 4 to particular images, such as red and black images or images of varying intensity using a head mounted display (HMD), as described below.

The computer system 20 includes the analysis module 30, a display module 32, an operating system 34 and a communications module 28 for receiving and transmitting signals over fixed or wireless connections. The computer system 20 may include the amplifier circuit 18, the ADC 20 and the display screen 22. The computer system 20 may include a standard computer 20 and the modules 28 to 34 may be software modules including computer program code. The standard computer may be a 64 bit Intel architecture computer produced by Lenovo Corporation, IBM Corporation, or Apple Inc, and, as described below, the processes executed by the computer system 20 are defined and controlled by computer program instruction code and data of software components or modules 28 to 32 stored on non-volatile (e.g. hard disk) storage of the computer 20. The operating system (OS) 34 may be Microsoft Windows, Mac OSX or Linux. The processes performed by the modules 28 to 32 can, alternatively, be performed by firmware stored in read only memory (ROM) or at least in part by dedicated hardware circuits of the computer 20, such as application specific integrated circuits (ASICs) and/or field programmable gate arrays (FPGAs). In particular, the process executed by the analysis module 30, which provides a neural event extractor 400, could be executed by dedicated digital signal processor chip, such as the C6000 DSP by Texas Instruments Inc. The processes can also be executed by distributing the modules 28 to 32 or parts thereof on distributed computer systems, e.g. on virtual machines provided by computers of a data centre.

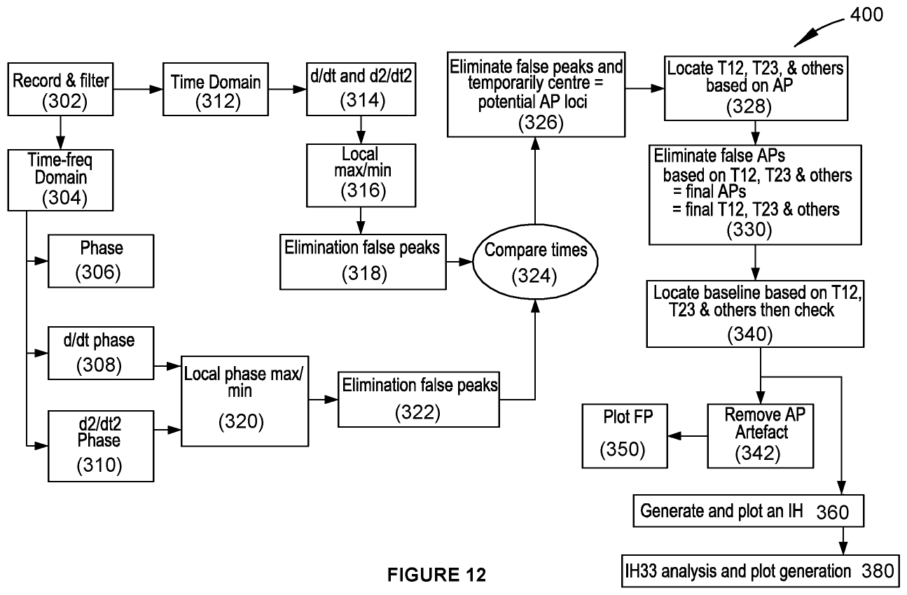

The vestibulo-acoustic response signal (FP) obtained from the patient is a composite signal that has been found to be produced primarily by the vestibular system and also comprising three or four timewise shifted and scaled presentations (pre-pre-, pre-, central-, post-) of afferent, efferent and vestibular nucleus (VN) major composite signal components that are each composed of one to three building block waveforms (EPSP, IAMFFP, EFP), described below, that can be decomposed to be used for both diagnosis and to determine drug efficacy. The VN has three components one from primary neurones in the VN, one from secondary neurones in the VN and one an inhibitory inverted EPSP (or IPSP) resulting from contralateral stimulation of the ipsilateral VN. There is also a broad long lasting inhibitory effect as a result of type II hair cell activation in the vestibular periphery suppressing post afferent responses. The analysis module 30 executes a neural event extraction process (NEEP) of the neural event extractor 400, as shown in FIG. 12, configured to determine specific loci in the composite signal to enable extraction of components that can focus on channels for depression, traumatic brain injury and also assist in discrimination between bipolar disorder (BD) and major depressive disorder (MDD).

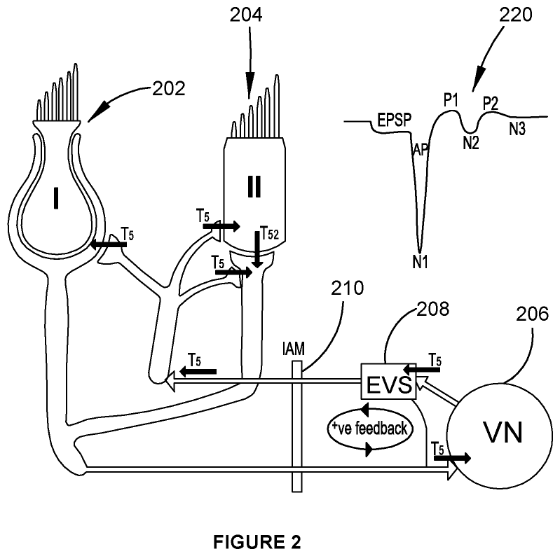

The vestibulo-acoustic composite signal obtained from the person 4 is an EVestG recording that effectively detects evoked or spontaneously evoked minute field potentials that can be processed by the neural event extractor 400 so as to produce a single averaged field potential (FP) waveform 220 that has a characteristic action potential (AP) point, as shown in FIG. 2. The system 2 is able to detect the minute FPs even when they are buried in noise using a model or template of the miniature FP waveform that is used to adjust the NEEP process 400 executed by the analysis module 30 to enable only the FPs to be extracted but also to focus on particular components and channels that relate to the vestibular system. The model for the average human FP waveform is described below and whilst the FPs may be acoustic, they are primarily generated by the vestibular system. The model uses one to three of the building blocks waveforms which are an Excitatory Post Synaptic Potential (EPSP) or an inverted version of this, i.e. an IPSP, an Extracellular Field potential (EFP) and an Internal Auditory Meatus Far Field Potential (IAMFFP) for each of three vestibular positive feedback loop components namely the afferent vestibular nerve, vestibular nucleus (VN) and efferent vestibular nerve, as described below.

The particularly high spontaneous firing rates of vestibular nerve fibres, their overlapping dendritic fields and the positive feedback loop imposed by the widely divergent efferent fields to hair cells (type II) and type I and II hair cell afferents, produce a ‘random’ occurrence of “synchronous firing” i.e. minute or miniature FPs.

There are actually 30,000 auditory nerve fibres and about 1600 efferents from the olivary complex projecting on to hair cells and their afferents. There are three auditory nerve spontaneous rate populations: high spike rate (>18 spikes/sec, typically 40-90 spikes/sec, mode around 60 spikes/sec), medium spike rate 0.5 to 18 spikes/sec and low spike rate <0.5 spikes/sec each being approximately 10, 15 and 75% respectively of the population. Comparatively, the resting spike rates of vestibular afferents is typically 70-100 spikes/sec. In primates there are 15,000 fibres in each vestibular nerve with about 100 action potentials/sec/nerve-fibre, so 1.5 million APs per second are present in the vestibular nerve (cf acoustic: 30,000×15spikes/sec (type I)=450,000 30% of the vestibular rate). If these APs were evenly distributed in time then in each 0.1 ms, 150 “almost synchronous” vestibular APs will appear as an FP. The AP distribution in nerve fibres across time is effected by other factors like past firing events, efferent input and local field potential spread with these potentially able to facilitate increased and decreased local spatial and temporal synchrony resulting in the generation of minute FPs.

Vestibular efferent effects in mammals are excitatory on all afferents and debatably excitatory (or inhibitory) on type II hair cells. Additionally, a smaller than anticipated level of Efferent Vestibular System (EVS) stimulation may be required given there are comparable impacts from both quantal (vesicular) and nonquantal (ascribable to intercellular K+ accumulation) transmission components. A comparison of auditory and efferent effects indicates:
Unlike the vestibular efferents, the auditory efferents from the Medial Superior Olive (MSO), when stimulated, reduce the compound action potential (CAP) N1 peak. Oppositely, auditory efferents from the Lateral Superior Olive (LSO) reduce the CAP when cut but do not effect threshold or latency. Vestibular efferents are mostly spontaneously active (10-50 spikes/sec) whereas most auditory efferents show no spontaneous activity.
Vestibular efferents participate in a fast, positive feedback loop with the greatest effects on central, irregular afferents which is accompanied by an increase in spike regularity. The periphery-VN-EVS-periphery feedback loop is shown in FIG. 2. The vestibular periphery hair cell (type I 202 and type II 204) to Vestibular Nucleus (VN) 206 to EVS 208 positive feedback loop is drawn for a dimorphic (Calyx and bouton connection to type I and type II hair cells (HCs) respectively) fibre. Efferents and afferents pass through the internal auditory meatus (IAM) 210 of the skull. Ts is the synaptic delay. On the type II hair cell (HC) 204 the efferent contacts the HC and afferent connection thus there are two latencies involved one from efferent to afferent and the other from efferent to hair cell to afferent. Only for larger stimuli is the HCII response evoked. The efferent connection to the type I HC is via the calyx afferent only. The waveform 220 is a single unitary (nerve fibre) potential indicating the regions (N1-3 and P1-2) which correspond to the FP formed from the sum of unitary potentials. The HC-VN-EVS positive feedback loop may propagate more than once before any centrally mediated inhibition is applied or a detectable FP is found. If so, the very small field potentials (FPs) may be further facilitated and or “synchronised” by efferent input. Yet when central inhibition is not present, extremely large vestibular firing rate fluctuations are observed. There is contralateral inhibition supplied to the ipsilateral VN on a regular basis determined by the latency of the efferent feedback loop and the signal intensity labelled VN contra. This signal is modelled to not evoke any EFP or IAMFFP component only an inverted EPSP. There is a primary and secondary VN response evoked by inputs from the afferents to the VN. The afferents connect to all four VN nuclei of which two directly connect (VNS and VNM) to the

EVS and two (VNI, VNL) connect first to secondary VN nuclei (VNS, VNM) then the EVS thus creating two VN responses VN1 and VN2. Both these waveforms consist only of EPSP and EFP components as they do not pass through the IAM so don't necessarily have an IAMFFP component.
In the vestibular, at least, the size and timing of synaptic potentials arriving at an afferent's spike-initiating zone might be effected by zonal variations in the larger scale dendritic morphology which can favour spatial and temporal averaging of EPSP's from multiple postsynaptic zones.

Accordingly, there is an underlying mechanism capable of generating a large number of minute APs which rather than being uniformly spaced in time appear modulated by inputs capable of effecting firing rate, shape and timing and collectively produce a modulated rather than uniform AP distribution across time. In a modulated distribution, the above mentioned 150 APs per 0.1 ms become both much larger and much smaller and the jitter associated with any distribution peaks is likely to cause any detected minute FPs to be slightly wider than that detected in response to an acoustic click evoked ECOG.

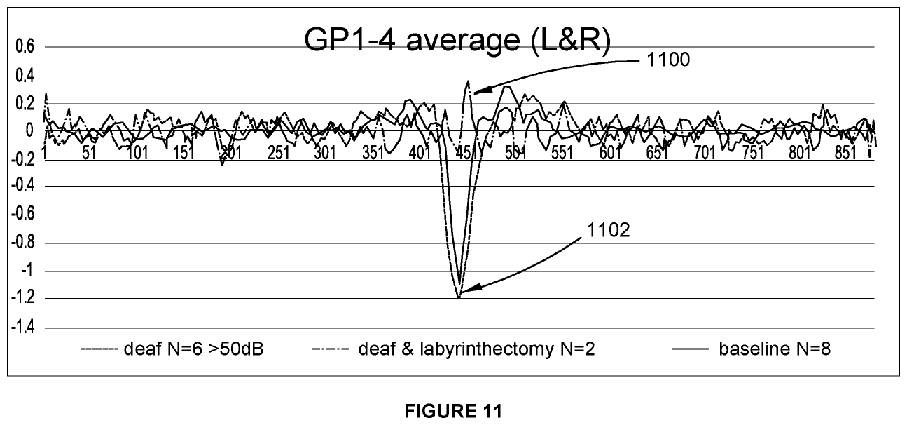

The above can be applied to the auditory afferents implying there should also be spontaneous minute auditory FPs detected as was observed following a chemical vestibular ablation with Gentamicin (unilateral weakness score 90%, sum of calorics 75%, slight high frequency hearing loss). The FP waveform produced 1100, as shown in FIG. 11, for the deafened and vestibular ablated animal subjects lacked the sharp AP produced by deaf only animal subjects 1102 or controls. The EVestG waveform is vestibulo-acoustic but the acoustic component is reduced relative to the vestibular component. There are major differences between acoustic and vestibular activity e.g. the spontaneous rate for cat type I auditory fibres is 15 spikes/sec compared with 50-100 spikes/sec for mammalian vestibular fibres. Additionally, the acoustic efferent system appears inhibitory compared to the excitatory positive feedback nature of the vestibular system. Thus, the vestibular FP waveform generation process described herein recognises that acoustic FPs will also be generated at minimum by a random process but may be considered largely inconsequential.

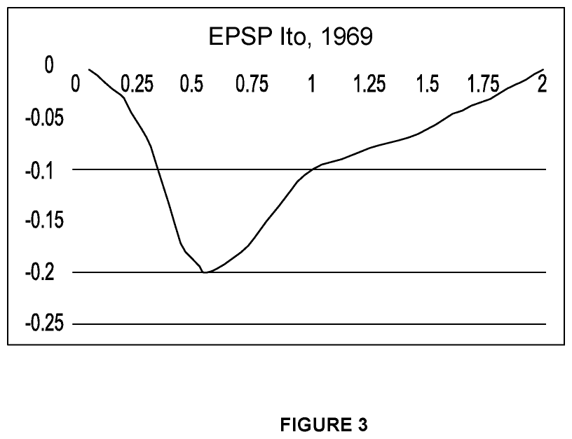

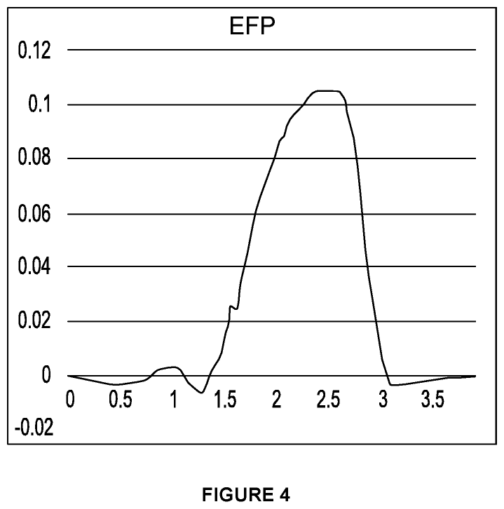

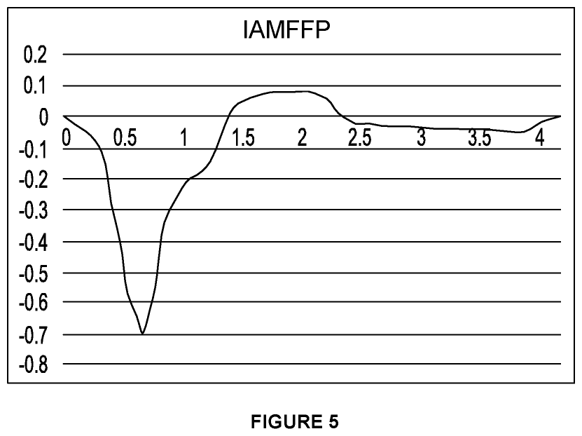

Stationary spontaneously evoked average FP response signals have been recorded by the system 2 from a population of human controls. To construct a simple model or template of the experimentally recorded FP, three vestibular components are used, namely the excitatory post synaptic potential (EPSP) (and its inverted version (IPSP)), as shown in FIG. 3 (horizontal axis time (ms), vertical axis voltage (mV)), extracellular field potential (EFP), as shown in FIG. 4 (horizontal axis time (ms) vertical axis voltage (mV)) and the human internal auditory meatus far field potential (IAMFFP), as shown in FIG. 5 (after near DC components were removed, horizontal axis time (ms) vertical axis voltage (mV)).
An EPSP is the change in membrane voltage of a postsynaptic cell following the influx of positively charged ions as a result of ligand sensitive channels. This depolarisation increases the likelihood of an AP. The effects can be cumulative. Additionally, K+ channels can modulate the shape of the EPSP and the AP. There are shorter (irregular inputs) and longer time (regular inputs) constant components of vestibular EPSPs. An IPSP can be modelled as the inverted EPSP.
EFPs are local extracellular field potentials (e.g. EEG). The higher frequency components of local FPs attenuate much more than lower frequency components distorting the FPs. In other words, the fast AP components (Na+) attenuate with distance whereas slower K+ components attenuate much less and these can be recorded, in the case of an EEG, over the scalp.
The IAMFP is a far-field or stationary potential, generated when the circulating action currents associated with each auditory neurone encounters a high extracellular resistance as it passes through the dura mater (at the IAM 210).

The model FP waveform using the above building blocks assumes as a starting point the simplest periphery-VN-EVS-periphery feedback loop of FIG. 2 and the latency information used is explained in Table 1 below which specifies the timing and scaling of each of the model components. It is important to note there are pre-pre-, pre-, central- and post-loop components representing up to four loops of the positive feedback efferent loop (shown in FIG. 2). Total simplified single loop time approximately 3.3 ms. This corresponds to the average experimentally observed time between detected EVestG FPs.

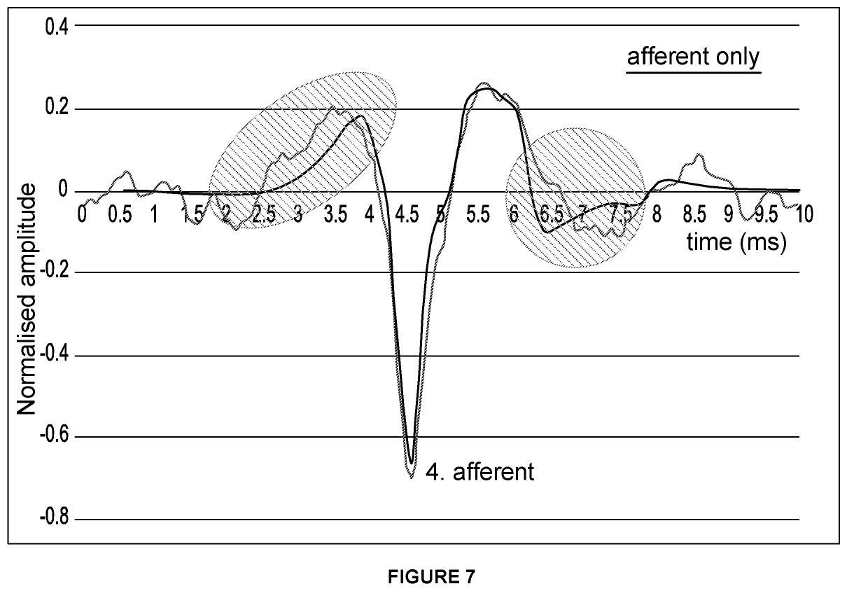

TABLE 1
Model Parameters FIG. 7

| | | N1 |
|---|---|---|
| Scale | | minima |
| Factor | name | (ms) |
| 1.0 | central-afferent | 4.46 |

| | | | | | | | | |
|---|---|---|---|---|---|---|---|---|
| | | N1 | | | | N1 | | Avg. |
| Scale | | minima | cycle | Scale | | minima | cycle | cycle |
| Factor | name | (ms) | (ms) | Factor | name | (ms) | (ms) | (ms) |

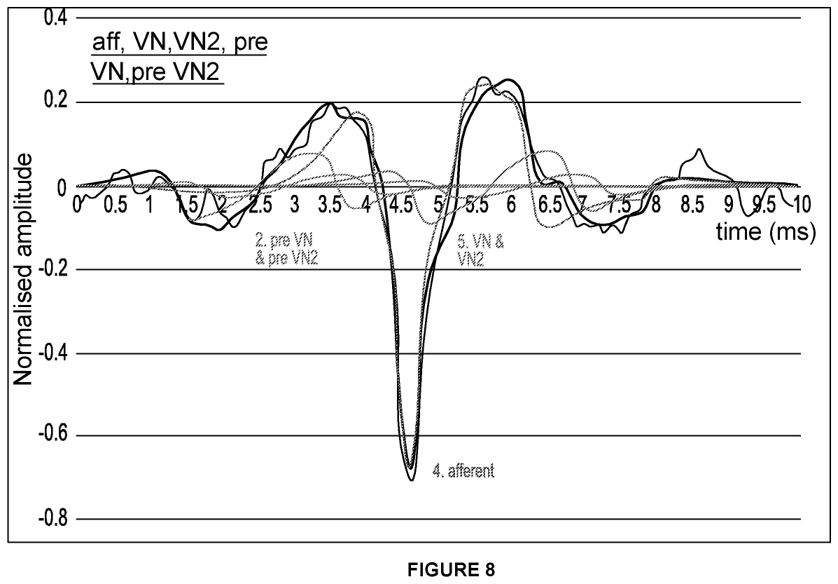

Model Parameters Plot of FIG. 8 (Plot of FIG. 7 plus pre- and central- VN1 and VN2)

| | | | | | | |
|---|---|---|---|---|---|---|
| | | | 1.00 | central-afferent | 4.46 | |
| 0.75 | pre-VN1 | 1.95 | 0.75 | central-VN1 | 5.30 | 3.35 |
| 0.25 | pre-VN2 | 2.35 | 0.25 | central-VN2 | 5.60 | 3.25 |

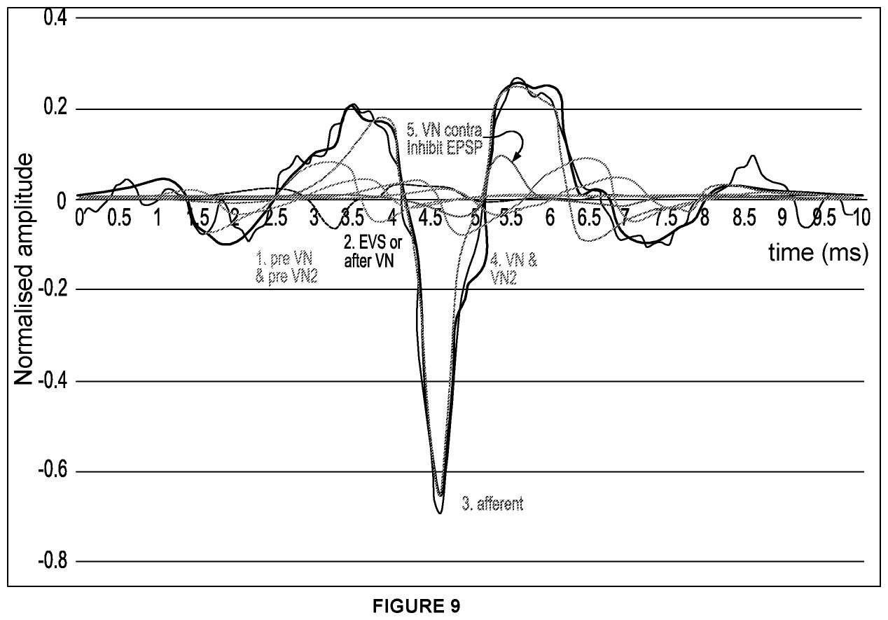

Model Parameters Plot of FIG. 9 (Plot of FIG. 8 plus pre-VN contra and central-EVS)

| | | | | | | |
|---|---|---|---|---|---|---|
| | | | 0.80 | central-EVS | 3.30 | |
| | | | 0.97 | central-afferent | 4.46 | |
| 0.75 | pre VN1 | 1.95 | 0.75 | central-VN1 | 5.30 | 3.35 |
| 0.35 | pre VN2 | 2.35 | 0.35 | central-VN2 | 5.60 | 3.25 |
| −1 | pre-VN contra | 5.50 | | | | |
| (EPSP) | | | | | | |

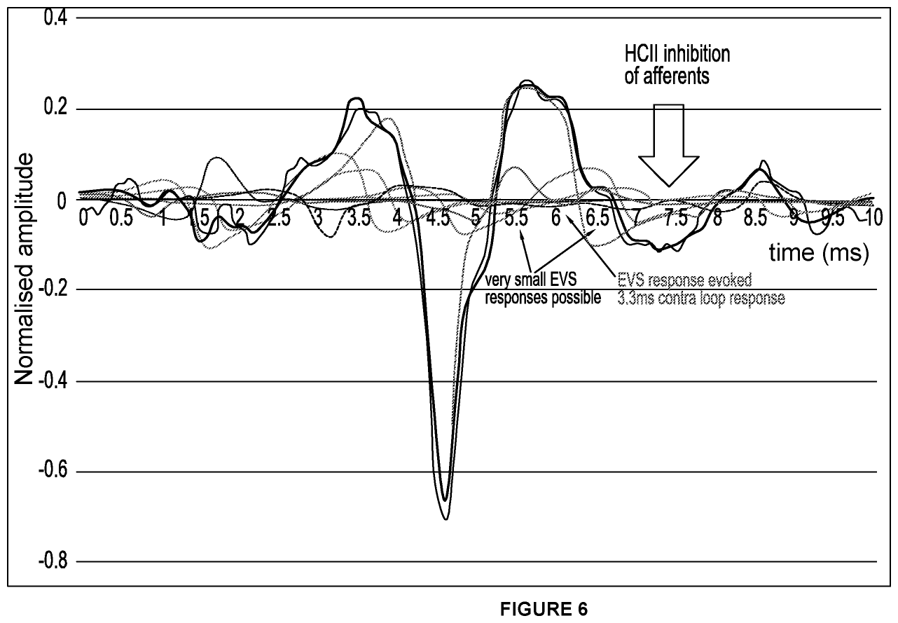

Model Parameters Plot of FIG. 6 (Plot of FIG. 9 plus pre-pre-VN contra, pre-afferent,
pre-VN contra, post-afferent, post-VN1 and post-VN2)

| | | | | | | | | |
|---|---|---|---|---|---|---|---|---|
| −1.00 | pre-pre-VN | 1.80 | | | | | | |
| | contra | | | | | | | |
| | | | | 0.88 | central-EVS | 3.20 | | |
| 0.06 | pre-afferent | 0.95 | | 0.93 | central-afferent | 4.46 | 3.51 | |
| 0.98 | pre-VN1 | 1.95 | | 0.64 | central-VN1 | 5.20 | 3.25 | |
| 0.63 | pre-VN2 | 2.15 | | 0.25 | central-VN2 | 5.60 | 3.45 | |
| −0.8 | pre-VN contra | 5.55 | 3.75 | −0.5 | central-VN contra | 8.70 | 3.15 | 3.45 |
| (EPSP) | | | | (EPSP) | | | | |
| | | | | 0.32 | central-EVS evoked | 6.60 | | |
| | | | | | central-VN contra | | | |
| | | | | 0.03 | post-afferent | 7.50 | 3.04 | 3.28 |
| | | | | 0.30 | post-VN1 | 8.90 | 3.70 | 3.48 |
| | | | | 0.20 | post-VN2 | 9.25 | 3.65 | 3.55 |

| | |
|---|---|
| Average Plot of FIG. 6 | 3.44 |

The IAMFFP is only present when the afferents or efferents pass through the internal auditory meatus (IAM) and not in the Vestibular Nucleus (VN) responses.

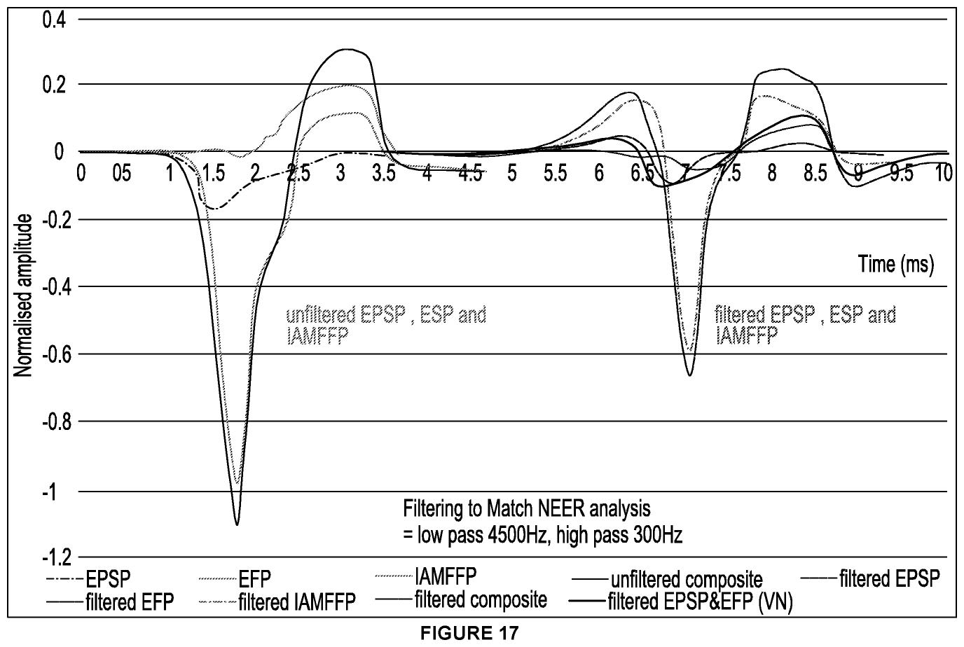

Considering first the central AP region only of the FP EVestG waveform (i.e. the ‘spontaneously’ evoked small FP in isolation), the major component of this region (referred to in Table 1) is the scaled afferent as shown in FIG. 7 of the feedback loop (i.e. only 1 of 3 possible anatomical contributors). The afferent waveform used was constructed as shown in FIG. 17 by combining the EPSP, IAMFFP and EFT components after each was filtered to match the NEEP processing. FIG. 17 compares the model (filtered composite line) incorporating the afferent (unfiltered EPSP, EFP and IAMFFP) with the average human experimentally recorded waveform (unfiltered composite line). The central AP region appears in FIGS. 6 to 10 centred around 4.6 ms. An average (from N=27 recordings) spontaneously evoked and EVestG recorded control FP from a human population 4 is overlaid in second thickest black line. The thickest black line trace or plot is the sum of the components used in the model. The scaling applied is to best match the control waveform (see Table 1).

In developing the model waveform, the VN components were added next to the model to improve matching in the shaded regions of FIG. 7. The positive feedback loop incorporates the VN, Efferent Vestibular System (EVS) and the vestibular periphery, and as shown in FIG. 8, the pre-VN1, pre-VN2, VN1 and VN2 components were added (see Table 1 for scaling and latencies). Pre-VN responses represent earlier activity in the feedback loop. As the VN does not project through the IAM there is no VN IAMFFP component in each of the VN1 and VN2 waveforms. The second thickest black line is the control average waveform (N=27) and the model matching this experimental result is markedly improved.

Similarly in FIG. 9, based on activity in the feedback loop, the central-efferent (black dashed line 2.) and pre-VN contra (inhibitory) responses have been added. The contralateral VN response (line 5.) is recognised as similarly effective in evoking efferent activity as the ipsilateral side (Table 1 provides the scaling and latencies). The second thickest black line waveform is the control average (N=27) and again the model matching the experimental result is markedly improved.

Third and fourth positive feedback loops that overlap the 0-10 ms window considered require incorporation of pre-pre-VN contra, post-(VN1, VN2, afferent) components. Additionally added components within the two loops already considered are the central-VN contra and pre-afferent (Table 1 provides the scaling and latencies). The second thickest black line is the control average (N=27) and the model matching the experimental result is further improved, as shown in FIG. 6.

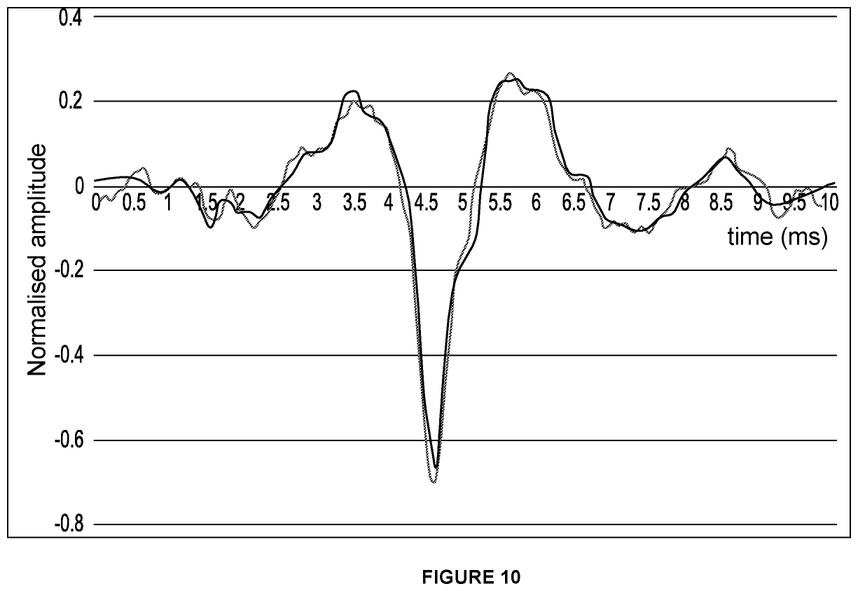

FIG. 10 shows an uncluttered comparison of the model or template (dark black line) and the FP waveform (second thickest line) generated by the system 2. The second thickest line being a human control plot is a display produced by the system 2 from a 1.5 sec static (no motion) segment response averaged across 27 human subjects 4.

As mentioned above, further confirmation that the EVestG waveform produced by the NEEP of the extractor 400 is vestibulo-acoustic and primarily due to the activity of the vestibular system is illustrated by the waveforms obtained from normal animals, deaf animals 1102 and animals 1100 that are deaf and have also been subject to vestibular ablation, as shown in FIG. 11.

Accordingly, based on the model, formation of the vestibular FP waveform is broken down into the following major components.

1. Formation of the pre-potential peak and preceeding region: This required inclusion of primarily the pre-VN1 and pre-VN2 components and secondarily, the pre-pre-VN contra, pre-afferent and efferent (EVS) response components.

2. Formation of the central regions of the FP: This required inclusion of components primarily the central-afferent, VN1, VN2 and pre-VN contra components and secondarily the EVS component.

3. Formation of the post-potential peak and following region: This required inclusion of primarily the central-VN contra (both EVS evoked (larger dash) and VN evoked), post afferent, post VN1 and post VN2 components and secondarily, the central afferent, central-VN1, and central-VN2 response components.

The efferents response is only visibly seen preceding the large central afferent response. The afferent response scaling varies widely, large centrally and quite small for preceding and following loops. The scaling for the 3 VN responses varies widely trending similarly to that for the afferent.

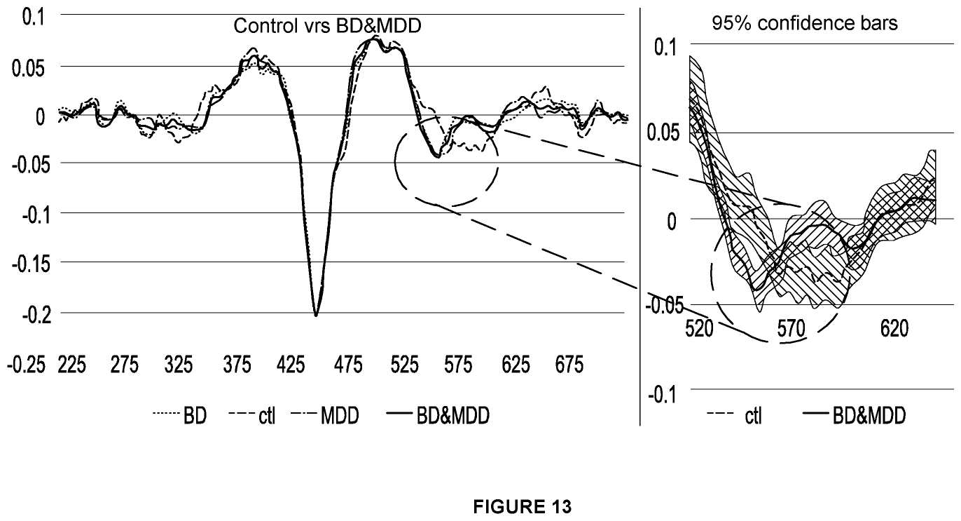

A number of the points on the curves of FIGS. 6 to 10 correspond to a sharp magnitude or sharp phase change in the composite FP waveform signal or the building block signals that form the composite signal. The NEEP of the extractor 400 is applied across the entire acoustic signal received from the patient 4 to detect all of the changes to provide better detection of the field potentials in noise and accordingly better determination of any pathological condition according to any deviation from the normal. For example, the changes are used to detect the sharp phase change (corresponding to a sharp local minima) that would occur, for example, at sample number 550 (circled) in FIG. 13 for depressed groups (i.e. T12 of FIG. 12) that can be related to the central-VN response and K+ ion channels. Similarly, any other abnormalities/differences (i.e. T23 points of FIG. 12) from the control/healthy waveform that result in local minima or maxima and their consequent phase changes can also be searched for and used to detect and identify specific pathologies such as mTBI or discern BD from MDD. NEEP is accordingly configured so as to detect those points of the components that correspond to the transitions and relate them via the FP building block waveforms to the vestibular system physiology such as ion channel mechanisms.

As discussed above, the three to four components from each of the passages around the EVS loop generating the composite FP signal shown in FIG. 10 are the pre-pre-, pre-central- and post-afferent, efferent and VN subcomponents each arising from distinct vestibular neural pathway regions and each built up from combinations of EPSP, IAMFFP and EFP building block waveforms.

Accordingly, based on the model, the components are:
Pre-pre-VN contra.
Pre-Afferent
Pre-VN1
Pre-VN2
Pre-VN contra
Central-EVS
Central-afferent
Central-VN1
Central-VN2
Central-VN contra
Central-EVS evoked Central-VN contra
Post-afferent
Post-VN1
Post-VN2

The NEEP of the extractor 400 is able to generate and locate characteristic loci for each of these components. It is also able to use them to determine to the loci corresponding to the important transitions in the FP waveform.

The neural event extraction process (NEEP) of the extractor 400, as shown in FIG. 12, uses known temporal and frequency characteristics of the FP waveform plot to try to accurately locate an evoked response from the patient 4. Latency between the points corresponds to a frequency range of interest.

The FP plot is known to exhibit a large phase change across a frequency range of interest at points on the FP plot, in particular, a T12 point, AP, onset and offset of AP and other T23 points. Additional to these are the points of maximum phase change associated with each of the components, described above, making up the composite waveform.

The neural event extraction process operates to produce a representative data stream that can be used to determine neural events occurring in the right time frame and with appropriate latency that can be considered to constitute characteristic parts of an evoked response or its building block waveforms. The same principle can also be applied to other vestibular (or auditory or visual) evoked responses as discussed below.

The neural event extraction process, as shown in FIG. 12, involves recording the voltage response signal output by the amplifier 18 in response to a head tilt (step 302) or when stationary. Where necessary a 50 or 60 Hz mains power notch filter is applied to the recording in the amplifier 18 to remove power frequency harmonics. The vestibulo-acoustic response signal from the amplifier 22 may also be bandstop and or high pass filtered (for example by a 300 Hz high pass 2 pole zero phase Butterworth filter or a filter to low pass recordings at 4500 Hz) to enable the extraction process to generate improved FP plots at step 350 for the display screen 22 or to remove noise. If the very low frequency data is retained, i.e. <50 Hz, then this can be used to plot (at step 350) discriminate efferent influences. An IH33 analysis 380 (described below) is also executed by the extractor 400. Absence or enhancement of the magnitude, latency or phase shifts compared to the model or template indicates a disorder.

The recorded response signal is decomposed in both magnitude and phase using a complex Morlet wavelet (step 304) according to the definition of the wavelet provided in equation (1) below, where t represents time, Fb represents the bandwidth factor and Fc represents the centre frequency of each scale. Other wavelets can be used, but the Morlet is used for its excellent time frequency localisation properties. The neural response signal x(t) is convolved with each wavelet.

$$ψ Morlet ( t ) = \frac{1}{\sqrt{2 π {F}_{b}}} {e}^{j 2 π {F}_{c} t - \frac{{t}^{2}}{2 {F}_{b}}} \qquad ( 1 )$$

To directly measure the vestibular system, seven scales are selected to represent wavelets with centre frequencies of for example 12000 Hz, 6000 Hz, 3000 Hz, 1500 Hz, 1200 Hz, 900 Hz and 600 Hz. Different frequencies can be used provided they span the frequency range of interest and are matched to appropriate bandwidth factors, as discussed below.

The wavelets extend across the spectrum of interest of a normal vestibular response signal 220, and also include sufficient higher frequency components so that the peaks in the waveform can be well localised in time. Importantly, the bandwidth factor is set to less than 1, being 0.1 for the scales representing 1500 to 600 Hz and 0.4 for all remaining frequencies.

Using a bandwidth factor that is so low allows for better time localisation at lower frequencies, at the cost of a frequency bandwidth spread, which is particularly advantageous for locating and determining neural events represented by the response signal. Magnitude and phase data is produced for each scale representing coefficients of the wavelets.

The phase data for each scale (306) is unwrapped and differentiated (308) using the “unwrap” and “diff’ functions of MATLAB. Any DC offset is removed, and the result is normalised for each scale to place it in a range from −1 to +1. This produces therefore normalised, zero average data providing a rate of phase change measurement for the response signal.

A first derivative of the phase change data (actually a derivative of a derivative) is obtained for each scale (308), and normalised in order to determine local maxima/minima rates of phase change (320). To eliminate any false peaks, very small maxima/minima are removed at a threshold of 1% of the mean absolute value of the first derivative (322).

All positive slopes from the first derivative (308) are set to 1, negative slopes to −1 and then a second derivative of the phase change data is obtained (310) to produce −2 and +2 step values. Each scale is then processed to look for resulting values of −2 and +2 which represent points of inflexion for the determined maxima and minima (320). For these particular loci, a value of 1 is stored for all scales. For the low frequency scale, i.e. 600 Hz, the actual times for both the positive and negative peaks are also stored for analysis to further isolate the responses as discussed below.

The original vestibulo-acoustic response signal in the time domain (312) is also processed to detect points which may be points of maximum phase change for comparative analysis with the extracted phase peaks from the wavelet analysis. Firstly, the mean and maximum of the original signal is determined. The signal is then adjusted to have a zero mean. Using this signal, the process locates and stores all points where the signal is greater than the mean minus 0.1 of the maximum in order to identify regions where an AP point is least likely (positive deviations above axis) and to exclude in later derivatives maxima as a consequence of noise. The slope of the original response is obtained by taking the derivative of the original response, and then also determining the absolute mean of the slope. For the result obtained, all data representing a slope of less than 10% of the absolute mean slope is set to 0. A derivative is then obtained of this slope threshold data (314) which is used to define the local maxima/minima of the slope (316).

Similarly, the absolute mean of this result is also obtained and a threshold of 10% of the mean used to exclude minor maxima/minima (step 318). All positive slopes of the original response are set to 1 and the negative slopes are set to −1, and then a second derivative obtained (314). From this derivative, each scale is examined to find values of −2 and +2, representing points of inflexion. The position of these loci are stored for the positive and negative peaks.

For each scale, if there is a positive peak, i.e. a maximum, determined from the first slope derivative, then for any peaks corresponding to these times (+1 or −1) these are set to 0 in any scale in which they appear in order to initially selectively look for the AP point which will be a minima.

The same is also done for points that were previously deemed unlikely regions for an AP point found during the original processing of the time domain response signal (312). The times of the peaks determined during processing of the phase data, and that determined during processing of the time domain signal, are compared (step 324). Because of scale dependent phase shifts inherent in detecting each wavelet scales phase maxima, the wavelet scale maxima are compared with those detected in the time domain and shifted to correspond to a magnitude minima in the time domain. Thus, potential AP loci (326) are determined.

The loci times for the low frequency scale, scale 7 representing 600 Hz, are searched to attempt to locate the additional T12 and or other points (T23), as it is most likely that the preceding steps have determined the AP point, due to the size of its appearance in the average signal and the difficulty of normally locating and or recognising the T12 and other non visually obvious points.

This search is undertaken over a range of interest for example proximal to sample 550 for depression in FIG. 13 looking for +2 values (i.e. negative peaks) in this range. If the value of the original response signal at the potential T12 point is close to −0.05, as shown in FIG. 13, then the potential T12 loci are stored for additional analysis.

If a T12 point is located, then the 600 Hz scale loci time for the T12 is stored. For verification, similar location procedures for the T12 point can be performed on the other scales, but this is not needed in all cases.

All of the scales are then processed (step 330) to look for maxima across the scales and link them to form a chain across as small a time band as possible. This allows false points, i.e. T12s, associated with all of the scales to be eliminated. The analysis module 28 is able to use a “Chain maximum-eliminate “false” maxima” routine of MATLAB® to perform this step.

As described below, a FP plot is formed by processing the time domain signal (or averaging the time domain signals obtained) centred on the local maxima determined previously. Maxima/minima values are further determined to establish the baseline (i.e. the average level before the response, as shown in FIGS. 2 and 10) and other points of interest (e.g. the dip at about 3.5 ms) depending on the pathology.

Using firstly the +2 values, and then the −2 values if no +2 values are found, for the points of inflexion determined from the phase data, the loci is searched (328) in the range allocated to the T12 previously determined. For each AP, remaining after all the elimination process (330) the T12 times are found and averaged to record a T12.

The T23 points of interest are found (328 and 330) similarly.

The baseline is found (340) by starting at a point −0.2 to −0.6 ms from the AP point (based on average FP shape), and again beginning with the +2 point inflexion values, and then −2 point inflexion values (if required) of the phase data in a time range initially allocated to the baseline. For each AP and other point of interest plus offset, the potential baseline times are found and averaged to record an initial baseline time. If the baseline time does not meet a baseline check, then the process is repeated starting with the new baseline time estimate. This process is repeated until a baseline check is met, which may be whether a baseline is within a pre-determined time range from the points. The average magnitude at the determined time is used. Alternatively, the baseline can be determined as being the mean of the first 300 samples of the FP.

An artefact, being a spike of about 3 samples wide, is produced at the tip of AP due to the selection of local minima in the time domain based on a scale determined loci proximal thereto. The samples corresponding to the spike (which may be up to 5 samples) should be removed, and this is done (342) by using the values of the points on either side of the spike to interpolate values into the removed sample positions. A filter, such as a 15 point moving average filter, can then be applied after removal to smooth the response.

Based on the points determined, which represent neural events, plots of the vestibular FP waveform response are generated and displayed (350). The plot is generated by the display module 30 using the times/loci of the maxima and minima determined by the neural event extraction process of the extractor 400.

To assist in reduction of noise, additional points/loci (which may be particularly relevant to pathologies such as depression) are included in the elimination process (330). This reduces the number of artefactual signals potentially mistaken as field potentials included in the averaging process. For example, the closing of the K+ channel (associated with the afferent potentials of the first major component) close to the 3 ms mark can assist in pathological discrimination. For example, in the Depression case the region indicated in FIG. 13 as significantly different can be included as a locus for detection of points of maximal phase change. This region would likely correspond to the orange VN waveform (and the closing of the K+ channels associated with this portion of that waveform). FIG. 13 is a plot of the average static (BGi) region field potential response for a left side recording showing the average responses of 27 Control, 43 Bipolar Disorder (BD) and 39 Major Depressive Disorder (MDD) patients. The horizontal axis is samples (41.67 samples/ms), and normalised amplitude is on the vertical axis. The black circles indicate overlaid 95% confidence regions illustrating the difference between the patients.

In summary, the neural event extraction process uses a complex time frequency approach with a variable bandwidth factor to determine the points where maximum/minimum phase changes occur across a range of frequencies characteristic of neural events associated with an FP plot. NEEP uses these and the component waveforms forming this composite signal.

The response signal is Morlet wavelet decomposed (i.e. an analysis of both magnitude and phase of the continuous signal) and the decomposition allows for the detection of potential neural events by first finding sharp changes in phase occurring almost simultaneously across all the wavelet frequency bands. These Morlet wavelet frequency bands are tailored further by adjusting their bandwidth factor to suit the signal (the lower and higher frequency bands are processed differently to obtain better temporal resolution particularly in the lower frequency bands). When potential neural events are found by the neural event extraction process they are compared against the FP waveform template and its building blocks to discern them from artefacts. This can be considered to involve:

(i) First a temporal template that detects whether the series of sharp magnitude and phase changes that occur timewise correspond to the AP, beginning and end of a real neural event.

(ii) Second a shape (relative magnitude) template that detects whether the series of sharp phase changes occur corresponding to the AP, beginning and end of the neural event.

If the potential neural event matches both in shape and time it is considered a neural event (and additionally its time of occurrence is stored for generating interval histogram timing data to produce an interval histogram (360)). It is then averaged with many other spontaneously or evoked neural events to produce a noise “free” or supressed field potential FP waveform that can be used for diagnostic or therapeutic purposes. The detected neural events are overlayed with the AP point being the centre of overlap for each detected neural event. The noise averages effectively to zero and the hidden neural events sum synchronously to appear as the FP.

The maximum/minimum phase change is used to establish the AP, T12, T23, baseline points and any other points detailed in the component or composite waveforms. Being able to determine these points enables elimination of other phase change events that are not related to an FP plot, such as those produced by background noise.

Also, maximum/minimum phase change points are correlated with events in the time domain to reduce time localisation error inherent in the use of the frequency domain representation provided by the wavelet analysis.

The system 2 as described is able to perform an accurate analysis of a response from the vestibule (and or vestibular brainstem and or EVS) that not only can be used for the detection of Meniere's disease, but can also be used for diagnosis of Parkinson's disease, post concussion syndrome (PCS), mild traumatic brain injury (mTBI) and depression as discussed below.

Also other neural events can be sought and determined, such as those produced by other vestibular and or auditory nuclei. The system 2 can be configured to obtain other Auditory Evoked Responses (AER) and the analysis module 30 used to accurately process the AER obtained, such as an ABR.

The system 2 is also able to detect changes associated with mild traumatic brain injury (mTBI), PCS and their respective medications/treatments. Both pathologies present with abnormal AP widths, known as the TAP measurement. The TAP is the baseline width of the AP and represents changes in Na+ channels. Similarly the AP area can be used.

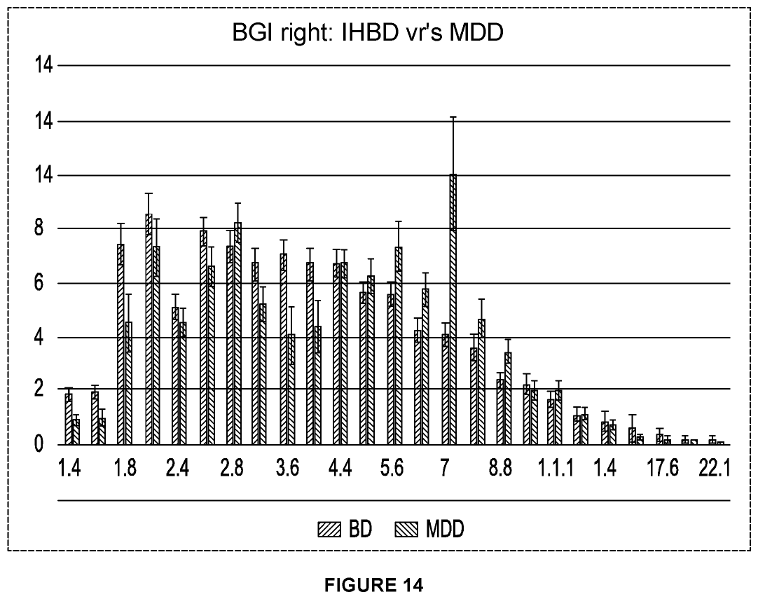

The system 2 is also able to detecting the decrease or increase in activity of cells projecting to the vestibular nuclei or a change in the local activity of either the VN or vestibular peripheral connecting cells in depression and other pathologies, as shown in FIGS. 13 and 14.

Accordingly, the system 2 is able to determine the major components (and their subcomponents) of a composite vestibulo-acoustic FP signal produced by the vestibular system of a person. For example, the pre-potential region dominated by pre-pre- and pre-components can be related to K+ and EVS differences between BD and MDD. The Central FP region relates to a sodium channel for traumatic brain injury and PCS. The post potential region dominated by post-components enables detection of Depression as in FIG. 13.

FIG. 14 shows the change in firing pattern in depression detected by the system 2. FIG. 14 is an interval histogram (IH33, gap between every 33^{rd} FP reflective of EVS activity) plot generated (360) for a right-side recording showing the average response of 43 Bipolar Disorder (BD) and 40 Major depressive Disorder (MDD) patients. There are 95% confidence error bars overlaid on each histogram bar. Interval time (ms) on a log scale is on the horizontal axis and the population % is on the vertical axis. There is a distinct discriminative shift to the right shift for MDD compared to BD.

This, like the post potential region components and its subcomponents, is particularly useful for objectively separating BD and MDD as well as quantitatively measuring the efficacy of therapies and drugs to treat depression.

For each field potential (FP) identified by the NEEP process, its time of occurrence is recorded, and the gap between each of the FPs is determined and the gaps are used to form the interval histograms (360). Diagnostic feature data is extracted from the interval histogram data (380) using the NEEP process which looks primarily at the gap between every 33rd field potential, i.e. the gaps between the 1st and the 33rd potentials, and the gaps between the 2nd and 34th potentials, and etc., effectively being the gap between field potential x and x+33. Other gaps could be used, e.g. 25 to 40. The interval was measured between every 33rd field potentials (approximately 100 ms) to measure low frequency modulations of the firing pattern as may be present if efferent modulation was present and labelled IH33. These intervals were calculated for the BGi (static phase) and onBB (deceleration phase after a tilt). The difference between the BGi and onBB interval histogram plots was determined and significant differences used to provide the data representing a diagnostic feature. This difference may be considered a measure of the dynamic response change in response to a vestibular input. It was compared (using a leave-one-out routine) for the BD plus MDD (depressed group) versus control groups. Average data for control versus depressed groups were found to have significant differences useful in characterizing populations. The IH33 region(s) with statistically significant difference between the average depressed and Control group responses were determined. Each significant bin was examined for robustness and the 95% significantly different bins (bin 84 minus bin 111) used to form the diagnostic feature data.

A vestibulo-acoustic response signal can be invoked using a head mounted display (HMD) worn by the patient 4 and connected to the computer system 2 or a separate computer running virtual reality software to drive and present images on the HMD. For example, a virtual reality (VR) environment consisting of a solid background was generated using the Unity Game Engine running on a laptop. Participants 4 were immersed in the VR environment by wearing a HMD (Oculus Rift, Development Kit 2) connected to the laptop (EUROCOM Sky X4, NVIDIA GTX 970M, G-Sync Technology). A sequence of colours (also at different intensities) (Black, White, Black, Blue, Black, Green, Black, Red, Black) were shown, when the participant 4 pushed a start button located on the chair 6. For each colour, the respective red, green and blue (RGB) value was initially set to 255 and the two others set to zero. Duration of each colour exposure was 30 seconds, and the recording lasted for 270 seconds. Presenting black background in between other colours was chosen to remove the image after effect.

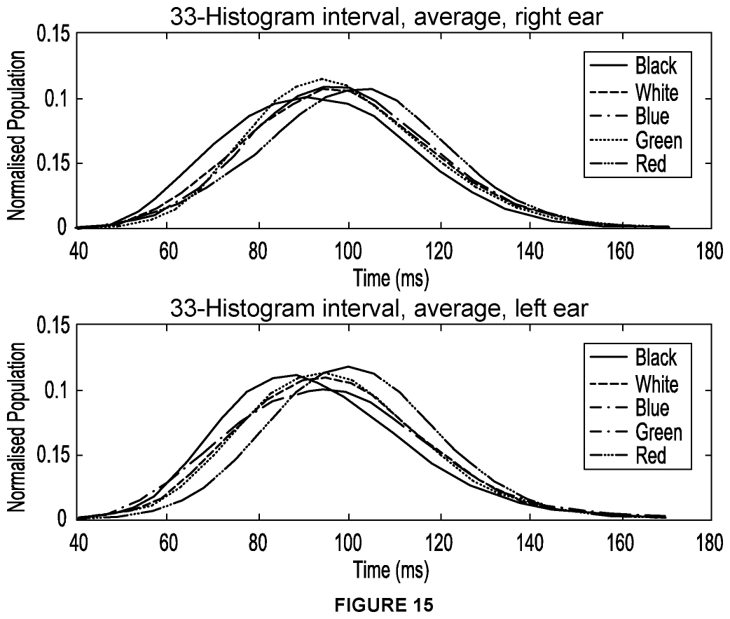

FIG. 15 shows the average of the IH33 histogram intervals for all participants for each ear generated (380) by the system 2. Based on the diagram, a range of vestibular responses was obtained following exposure to light of different colours. The largest difference corresponded to the black and red histograms (p<0.1 for both ears) with the black colour having the shortest average interval. Accordingly, the images presented on a HMD produce different vestibular responses, and allow responses to be invoked based on a VR environment presented without any movement of the patient 4.

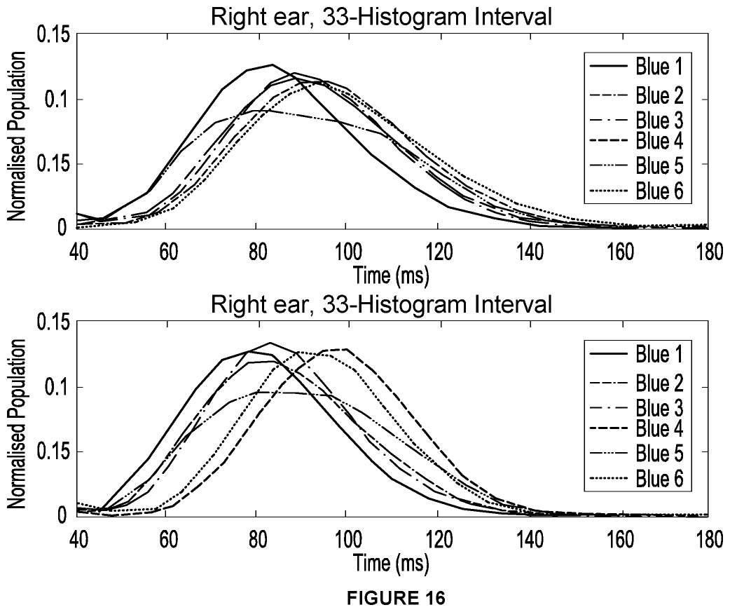

Also shown in FIG. 16 is the effect of different blue color intensities on the IH33 histogram. Blue 1 to Blue 4 lines are for the photopic region, and Blue 5 to Blue 6 lines the mesopic region.

Many modifications will apparent to those skilled in the art without departing from the scope of the present invention as defined in the claims and described herein with reference to the accompanying drawings.

1. A method for processing a vestibulo-acoustic signal, including:
receiving an vestibulo-acoustic signal obtained from a person;
decomposing said signal using wavelets;
differentiating said signal and phase data of said wavelets to determine loci of components of a composite field potential waveform produced by the vestibular system of the person.

2. A method as claimed in claim 1, wherein the components comprise at least one of a pre-pre-component, a pre-component, central-component and post-component associated with a vestibular system positive feedback loop.

3. A method as claimed in claim 2, wherein the pre-component discriminates between bipolar disorder (BD) and major depressive disorder (MDD).

4. A method as claimed in claim 2, wherein the central-component corresponds to a sodium channel associated with traumatic brain injury and/or PCS.

5. A method as claimed in claim 2, wherein the central-component and post-component correspond to a potassium channel associated with depression.

6. A method as claimed in claim 2, wherein the post-component discriminates between bipolar disorder (BD) and major depressive disorder (MDI)).

7. A method as claimed in claim 2, wherein the pre-pre-component and pre-components include central-afferent, pre-VN1, pre-VN2 and ENS response subcomponents and each comprising excitatory post synaptic potential (EPSP), extracellular field potential (EFP) and internal auditory meatus far field potential (IAMFFP) component waveforms.

8. A method as claimed in claim 2, wherein the central-component includes central-afferent and EVS, VN1 and VN2 response subcomponents and each comprising vestibular efferent excitatory post synaptic potential (EPSP), extracellular field potential (EFP) and internal auditory meatus far field potential (IAMFFP) component waveforms

9. A method as claimed in claim 2, wherein the post-component includes central-afferent, central-VN1, pre-VN contra response subcomponents and each comprising excitatory post synaptic potential (EPSP), extracellular field potential (EFP) and internal auditory meatus far field potential (IAMFFP) component waveforms.

10. A method as claimed in claim 8, wherein the central afferent IAMFFP subcomponent indicates mTBI and/or PCS in said person.

11. A method as claimed in claim 8, wherein the central VN1 subcomponent indicates depression in said person

12. A method as claimed in claim 8, wherein the central VN1 and central VN2 subcomponents indicates BD or MDD in said person

13. A method as claimed in claim 1, including generating interval histogram data associated with field potentials of said composite waveform, and the intervals between every 33rd or every one of 25-40 field potential are used in discriminating between bipolar disorder (BD) and major depressive disorder (MDD).

14. A method as claimed in claim 1, wherein said decomposing is performed using wavelets with a bandwidth factor less than one.

15. A method as claimed in claim 1, wherein said wavelets have centre frequencies across a frequency spectrum of said signal.

16. A method as claimed in claim 1, wherein said differentiating includes generating a number of derivatives of said phase data produced by said decomposing, and said loci represent rates of change of phase of scales of said wavelets.

17. A method as claimed in claim 1, wherein said differentiating includes generating a number of derivatives of said signal to produce said loci, and said processing includes correlating said loci of said phase data and said signal based on a template for said waveform.

18. A method as claimed in claim 1, including generating data indicating whether said person has a disorder.

19. A computer system for executing the method as claimed in claim 1.

20. A computer readable medium having computer program code for use in performing the method as claimed in claim 1.

21. A vestibulo-acoustic signal processing system, including:
electrodes for connecting to a person to obtain a vestibulo-acoustic signal; and an analysis module for decomposing said signal using wavelets, and differentiating said signal and phase data of said wavelets to determine loci of components of a composite field potential waveform produced by the vestibular system of the person.

22. A system as claimed in claim 18, wherein the electrodes are cotton wool tipped electrodes with lead wires wrapped with shielded coaxial cable.

23. A system as claimed in claim 18, wherein one of said electrodes is placed at least adjacent a tympanic membrane of the person.

24. A system as claimed in claim 18, including a head mounted display presenting images to invoke said signal.

25. A system as claimed in claim 18, wherein the person is in a supine position.

26. A vestibula-acoustic signal processing system, including:
electrodes for connecting to a person to obtain a vestibulo-acoustic signal;
a head mounted display presenting images to invoke said signal; and
an analysis module for processing said signal to generate a field potential waveform produced by the person.
